(19) United States
(12) Patent Application Publication
(10) Pub. No.: US 2025/0011263 A1
(43) Pub. Date: Jan. 9, 2025

(54) A MODIFIED AIR STRIPPING PROCESS AND APPARATUS

(71) Applicant: ANDRITZ OY, Helsinki (FI)

(72) Inventors: Naveen CHENNA, Helsinki (FI); Otto GREIS, Helsinki (FI); Aino PESOLA, Helsinki (FI); Pekka TERVOLA, Helsinki (FI)

(21) Appl. No.: 18/711,524

(22) PCT Filed: Nov. 17, 2022
(86) PCT No.: PCT/FI2022/050758

§ 371 Date: May 17, 2024

(30) Foreign Application Priority Data
Nov. 18, 2021 (FI) 20216184

(51) Int. Cl. C07C 29/48; C07C 29/78

(52) U.S. Cl. CPC C07C 29/48; C07C 29/78

(57) ABSTRACT

A process wherein lignin is oxidized into methanol in an air stripping column. The process includes: feeding a feedstock into the air stripping column, wherein the feedstock includes lignin such as a filtrate from a pulp mill; and feeding air into the air stripping column to oxidize methoxyl groups of the lignin into methanol.

### TECHNICAL FIELD

The present disclosure generally relates to production of methanol in a modified air stripper. The disclosure relates particularly, though not exclusively, to a process and system for producing methanol in an air stripper by using lignin containing side streams produced in pulp mills.

### BACKGROUND

This section illustrates useful background information without admission of any technique described herein representative of the state of the art.

Air stripping is typically used to remove volatile compounds from liquids. In air stripping air acts as a carrier gas for volatile compounds present in the liquid. When air is blown through the liquid, volatile compounds, such as methanol, present in the liquid moves from the liquid phase into the carrier gas and can be removed together with the carrier gas. In conventional air stripping methods, the purpose of the process is to separate volatile compounds from the liquid, such as from processed liquid feedstocks.

It is an object to develop a method which enhances utilization of filtrates by converting lignin present in filtrates into methanol.

### SUMMARY

The appended claims define the scope of protection. Any example and/or technical description of an apparatus, system, product and/or method in the description and/or drawing which is not covered by the claims, is presented herein not as an embodiment of the invention but as background art or example useful for understanding the invention.

It is an object of the present disclosure to provide a method for producing methanol from lignin present in waste streams and side streams originating from industrial processes, such as from filtrates and black liquor produced in pulp mills. Another object is to provide an alternative solution to existing technology in methanol production and/or in lignin processing.

According to a first aspect is provided a process for producing methanol in an air stripper comprising:
i. feeding into an air stripping column a feedstock comprising lignin; and
ii. feeding into the air stripping column air to oxidize methoxyl groups of the lignin into methanol.

The present process differs from conventional stripping methods for example in that conventional air stripping methods only aim to transfer volatile compounds dissolved in a liquid phase into the carrier gas. In the present process oxygen present in the carrier gas (air) is utilized to oxidize lignin of the feedstock fed into the air stripping column to produce methanol, which then transfers to the carrier gas.

In an embodiment the feedstock is a lignin-containing side stream of a pulp mill.

In an embodiment the feedstock is a filtrate produced in a pulp mill.

In an embodiment an oxidation booster selected from oxygen, ozone, hydrogen peroxide, or any combination thereof is additionally fed into the air stripping column to enhance oxidation of the lignin.

In an embodiment the air stripping column is operated at a pH value selected from the range 5-14, preferably at 8-13, more preferably at 10-12, and most preferably at about 11. In a preferred embodiment pH is controlled to the selected range during the process. Oxidation of lignin decreases pH, and adding alkali can be used to keep the pH in the selected range, thereby driving the reaction to increased oxidation of lignin, and into increased production of methanol.

In an embodiment the air stripping column is operated at a temperature selected from the range 60-200° C., preferably 70-150° C., more preferably 80-120° C., most preferably 90-160° C. or 90-100° C. Temperatures in the lower end of the above ranges may be preferable when it is desirable to use less energy in the process. Temperatures in the higher ends of the above ranges may be preferable to enhance evaporation of methanol and to increase methanol production.

In an embodiment the air stripping column is operated at a pressure selected from the range 0.25-25 bar(g), preferably 1-20 bar(g), more preferably, 2-10 bar(g), more preferably 4-6 bar(g), most preferably at about 5 bar(g). Pressures in the higher ends of the above ranges may be useful to increase production of methanol, because a higher pressure allows feeding more oxygen to the process.

In an embodiment oxidized feedstock is transferred to fiber line circulation of a pulp mill, or to an evaporation plant. In a preferable embodiment in such a case the feedstock is fiber line filtrate.

In an embodiment methanol vapor produced in the air stripping column is transferred to a condenser unit to liquefy methanol, and then to a methanol purification unit. In an embodiment the methanol purification unit is a distillation column configured to separate methanol.

According to a second aspect is provided a system comprising means for performing the process of the first aspect or any of its embodiments.

### BRIEF DESCRIPTION OF THE FIGURES

Some example embodiments will be described with reference to the accompanying figures, in which:

FIG. 1 schematically shows as an example embodiment certain parts of a system configured to carry out the present process.

### DETAILED DESCRIPTION

In the present description like reference signs denote like elements or steps.

In the present disclosure Adt refers to air dry ton.

In the present disclosure COD refers to chemical oxygen demand expressed as mg/l. The COD can be determined according to ISO 6060:1989 Water quality—Determination of the chemical oxygen demand.

As used herein, the term “comprising” includes the broader meanings of “including”, “containing”, and “comprehending”, as well as the narrower expressions “consisting of” and “consisting only of”.

In an embodiment the process steps are carried out in the sequence identified in any aspect, embodiment, or claim. In another embodiment any process step specified to be carried out to a product or an intermediate obtained in a preceding process step is carried out directly to said product or intermediate, i.e. without additional, optional or auxiliary processing steps that may chemically and/or physically alter the product or intermediate between said two consecutive steps.

In an embodiment the present process is an industrial process. In another embodiment the industrial process may exclude small scale methods such as laboratory scale methods that are not scaled up to volumes used in industry.

In embodiment the feedstock is liquid feedstock.

In an embodiment the air stripping column is operated in conditions in which at least methanol is in gaseous phase, and in which water is in liquid phase. The skilled person can determine for example the operating temperature and pressure of the air stripping column to achieve these objectives.

In an embodiment lignin present in the feedstock is at least partially dissolved or solubilized.

Lignin contains methoxyl groups that can be converted to methanol by the present process. Lignin is either nonphenolic or phenolic, and approximately 40% of lignin is phenolic and 60% is non-phenolic. Phenolic lignin is reactive to mild oxidative conditions whereas non-phenolic lignin requires more oxidative conditions and more intense environment to produce methanol. With the operating conditions and the oxidative agent disclosed herein, methoxyl groups of phenolic and non-phenolic lignin available in the lignin structure can be converted to methanol.

In an embodiment pH is adjusted to a value selected from the range 5-14. The adjustment can be made by feeding any alkali or acid into an inlet of recirculation pump which is configured to recirculate the fluid inside the stripping column. In another embodiment the alkali or acid is directly fed into a feed tank containing the feed to be processed in the stripping column. The pH adjustment can be made initially at the beginning of the oxidative treatment, and/or during the lignin oxidation reaction to keep the pH at or near the selected pH value.

In an embodiment NaOH is used to adjust pH.

In an embodiment non-condensable gases are removed by a non-condensable gas handling system.

In an embodiment the feedstock comprises brown stock washing filtrate.

In an embodiment the feedstock comprises black liquor, such as weak black liquor. Softwood black liquors, hardwood black liquors, and their mixtures can be used in the present process.

In an embodiment the feedstock comprises post oxygen washing filtrate.

In an example embodiment the feedstock comprises filtrate from wood pulping, such as from hardwood pulping. In an embodiment the filtrate has at least one component present in an amount shown in Table 1, or within an error margin of 15% of any amount show in Table 1.

TABLE 1
Hardwood filtrate compositions.

| | | Post oxygen | Post oxygen |
|---|---|---|---|
| | Brown stock | washing filtrate | washing filtrate |
| | washing filtrate | (no cleaning) | (clean) |
| pH | 10-11 | 10-11 | 10-11 |
| COD, mg/l | 81600 | 19400 | 15400 |
| Lignin, mg/l | ~10000 | ~4000 | ~4000 |
| S, mg/l | 3 190 | 2 090 | 1 600 |

In an embodiment the air stripping column is operated such that the oxidation reactions are carried out up to 100 min, such as 10-100 min, 20-100 min, 30-100 min, 50-100 min or about 100 min.

In an embodiment non-condensable gases are removed by a non-condensable gas handling system in fluid communication with the air stripping column.

In a preferable embodiment the air stripping column is operated at a temperature of 90-100° C., pH 8-14, pressure of about 5 bar for up to 100 min and by using oxygen as the oxidation booster.

In conventional air strippers air is not used in the amounts that are used in the present process to oxidize lignin in the air stripper. The amount of air used in the conventional air strippers is selected to be just sufficient to remove a wanted compound, such as methanol, in the selected operating conditions and in the desired purity, and use of any excess air should therefore be avoided. In conventional air stripping the amount of air is therefore desirable to be as low as possible.

In an embodiment of the present invention, in addition to the amount of air, which is sufficient to strip methanol from the feedstock, an additional amount of air or oxidant booster is fed into the stripper. In an embodiment the amount of additional air or oxidation booster fed is at least 30 mass-% more than the amount which is sufficient to remove methanol from the feedstock fed into the air stripper. In another embodiment the amount of additional air or oxidation booster is at least 30 mass-%, 50 mass-%, 100 mass-%, 200 mass-%, 500 mass-%, 1000 mass-%, 1300 mass-% or 1500 mass-% or 2000 mass-% more. Methanol can be considered to be removed from the feedstock in an air stripper when 90 mass-% of it is removed. In an embodiment the amount of air which is required to remove 90 mass-% of methanol in the stripper is determined, and the amount of additional air and/or oxidation booster is calculated based on this determined amount.

In an embodiment the oxidation booster is gaseous.

In an embodiment the amount of the oxidation booster, preferably oxygen, used in the oxidative reactions is not more than 12 mass-% of the filtrate DS.

In an amount the amount of oxidation booster, preferably ozone and/or hydrogen peroxide, used in the oxidative reactions is not more than 24 mass-% of the filtrate DS.

In an embodiment the oxidation booster is air, and it is used in an amount which is not more than 55 mass-% of the filtrate DS.

In an embodiment air and the oxidant booster are fed into the air stripping column through a nozzle or an inlet port arranged inside the air stripping column. In an embodiment the oxidant booster is fed through a separate nozzle or inlet.

In an embodiment the amount of air fed into the air stripping column is controlled such that it is sufficient to remove methanol from the aqueous feedstock into gaseous form to be mixed with the air, and an oxidation booster is fed into the air stripping column to oxidize lignin, In this embodiment the amount of air does not necessarily need to be higher than used in conventional air stripping, because the production of methanol is achieved by the added oxidation booster.

In an embodiment the oxidized feedstock is transferred back to fiberline of the pulp mill after the oxidative air stripping.

In a further alternative or additional embodiment, methanol is recovered in a methanol column in fluid connection with the top part of the air stripping column. In an embodiment the methanol column is directly in fluid connection with the air stripping column.

In an embodiment the present process is a continuous process. A continuous process can be integrated into the process of the pulp mill.

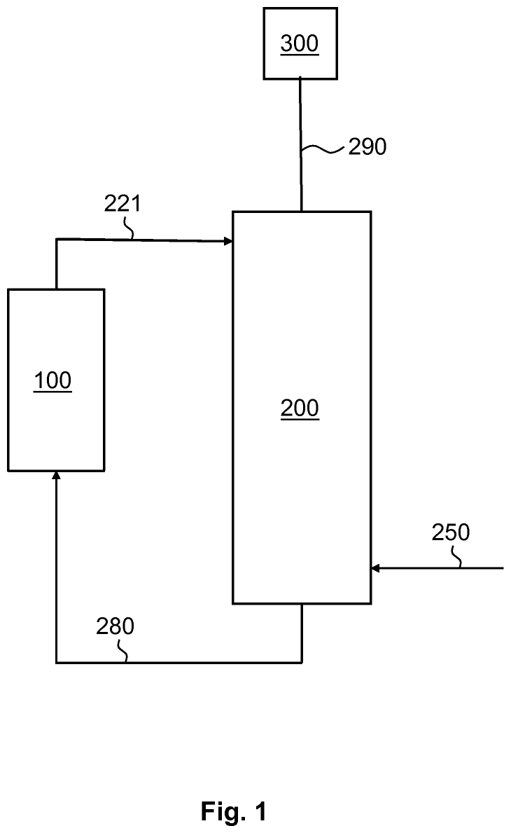

An example embodiment disclosing certain parts of a system configured to carry out the present process is illustrated in FIG. 1, in which line 221 is configured to feed liquid feedstock from the fiberline 100 into the top part of the air stripping column 200.

To the air stripping column 200 is connected an inlet line 250, which can be configured to feed the air, and optionally an oxidant booster, into the air stripping column 200. In an alternative or additional embodiment the oxidant booster is fed into the air stripping column 200 via another inlet line not shown in FIG. 1.

Oxidized filtrate, from which volatile compounds such as methanol are removed by the present air stripping process, is conducted back to the fiberline 100 through an outlet line 280 connected to the bottom of the air stripping column 200.

Air and other gaseous and volatile compounds are removed from the column via air stripping column outlet line 290 arranged on the top part of the air stripping column 200. The air stripping column outlet line 290 can be configured to conduct the gases to a methanol purification unit 300.

The inlet and outlet lines that are configured to transfer material and are shown in FIG. 1, can be equipped with one or more valve and one or more pump to allow better control of the process, and to ensure efficient transfer of gaseous and liquid phases in different parts of the system. Sampling points can be arranged in pipes or vessels of the system to allow analysis of the material in the process, as well as to determine other process parameters.

Heating/cooling means and/or pressure control means can be arranged in the air stripper to control the operating temperature and pressure.

### Examples

In an example embodiment the present process was carried out to a filtrate obtained from a pulp mill at the operating conditions disclosed in Table 2. The results show that the present process was successful in producing methanol by oxidizing lignin present in the filtrate.

TABLE 2
Example conditions for filtrate air stripping.

| | Unit | Value |
|---|---|---|
| Temperature | ° C. | 90-160 |
| pH range | | 8-14 |
| Optimal pH | | 11 |
| Pressure | bar | 5 |
| Oxidant | | air |
| Oxidant booster | | oxygen |
| Reaction time | min | 0-100 min |
| Raw material | liquid/gas | liquid |
| Methanol yield from O₂ filtrate | kg/Adt | 2.5 |

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A process for producing methanol in an air stripper comprising:
feeding into an air stripping column a feedstock comprising lignin;
feeding into the air stripping column air,
oxidizing in the air stripping column methoxyl groups of the lignin using the air in the air stripping column; and
generating methanol from the oxidation of the methoxyl groups.

2. The process of claim 1, wherein the feedstock is a lignin-containing side stream of a pulp mill.

3. The process of claim 1, wherein the feedstock is a filtrate produced in a pulp mill.

4. The process of claim 1, wherein an oxidation booster selected from at least one of oxygen, ozone, hydrogen peroxide, or any combination of the oxygen, the ozone and/or the hydrogen peroxide is additionally fed into the air stripping column to enhance the oxidation of the methoxyl groups in the lignin.

5. The process of claim 1, wherein the air stripping column is operated at a pH value in a range of 5 to 14.

6. The process of claim 1, wherein the air stripping column is operated at a temperature in a range of 60° C. to 200° C.

7. The process of claim 1, wherein the air stripping column is operated at a pressure in a range of 0.25 bar(g) to 25 bar(g).

8. The process of claim 1, further comprising transferring oxidized feedstock from the air stripping column to a fiber line circulation in a pulp mill.

9. The process of claim 1, wherein the methanol is at least partially methanol vapor and the process further comprises:
transferring the methanol vapor from in the air stripping column to a condenser unit;
condensing the methanol vapor in the condenser unit to liquefied methanol, and
transferring the liquefied methanol to a methanol purification unit.

10. (canceled)

11. A process comprising:
feeding a feedstock including lignin into an air stripping column;
feeding air into the air stripping column,
using the air to oxidize methoxyl groups of the lignin in the feedstock in the air stripping column, and
generating methanol from the oxidization of the methoxyl groups.

12. The process of claim 11, further comprising transferring wherein the feedstock is a lignin-containing side stream of a pulp mill.

13. The process of claim 11, further comprising providing a filtrate produced in a pulp mill as the feedstock fed into the air stripping column.

14. The process of claim 11, further comprising feeding an oxidation booster into the air stripping column and using the oxidation booster to assist in the oxidization of the methoxyl groups, wherein the oxidation booster includes at least one of oxygen, ozone or hydrogen peroxide.

15. The process of claim 11, further comprising operating the air stripping column at a pH value in a range of 5 to 14 while the air oxidizes the methoxyl groups.

16. The process of claim 11, further comprising operating the air stripping column such that the feedstock in the air stripping column is at a temperature in a range of 60° C. to 200° C. while the air oxidizes the methoxyl groups.

17. The process of claim 11, further comprising operating the air stripping column such that the feedstock in the air stripping column is under a pressure in a range of 0.25 bar(g) to 25 bar(g) while the air oxidizes the methoxyl groups.

18. The process of claim 1, further comprising transferring oxidized feedstock from the air stripping column to a fiber line circulation in a pulp mill.

19. The process of claim 1, wherein the methanol generated in the air stripping column is at least partially methanol vapor, and the process further comprises:
transferring the methanol vapor from the air stripping column to a condenser unit;
condensing the methanol in the condenser unit to produce liquified methanol, and
transferring the liquefied methanol to a methanol purification unit.
